(19) United States
(12) Patent Application Publication
(10) Pub. No.: US 2012/0065666 A1
(43) Pub. Date: Mar. 15, 2012

(54) SEAL-LESS DEVICE FOR DISPENSING MICROLITER QUANTITIES OF A MATERIAL INTO A SITE

(75) Inventors: James William Ashmead, Middletown, DE (US); William Gerald Dimaio, JR., Boothwyn, PA (US); Grant L. Vincent, Wilmington, DE (US)

(73) Assignee: E. I. DU PONT DE NEMOURS AND COMPANY, Wilmington, DE (US)

(21) Appl. No.: 12/879,138

(22) Filed: Sep. 10, 2010

(51) Int. Cl. A61B 17/03

(52) U.S. Cl. 606/213

(57) ABSTRACT

A seal-less, hand-held dispensing device includes a support platform having a pair of finger gripping surfaces; first and second hollow members on the platform; and a compatibly sized plunger disposed in each hollow member. Each hollow member has a largest inside dimension in the range from about 0.2 mm to about 0.3 mm and each plunger has a largest outside dimension that is sized for compatible receipt within a hollow member. The largest outside dimension of each compatibly sized plunger is from about eighty percent (80%) to about ninety-five percent (95%) of the largest inside dimension of a hollow member. An actuator having a thumb actuating surface thereon is operatively engagable with each plunger. When fully extended a predetermined maximum finger span is defined between the actuating surface and the gripping surfaces, the maximum finger span being not greater than about 150 mm.

### CROSS-REFERENCE TO RELATED APPLICATIONS

Subject matter disclosed herein is disclosed and claimed in the following copending applications, all filed contemporaneously herewith and all assigned to the assignee of the present invention:

Device For Dispensing Microliter Quantities Of A Material Into A Longitudinally Extending Wound Site (CL-4857); and

Device For Dispensing Microliter Quantities Of A Material Into A Puncture Wound Site (CL-5078).

### BACKGROUND OF THE INVENTION

1. Field of the Invention

This invention relates to an apparatus used for dispensing a material into a site, such as the dispensation of a fast-setting multi-component medical adhesive into a wound site.

2. Description of the Prior Art

Infliction of a wound on the cornea or the sclera of the eye is a necessary consequence of most ophthalmic surgical procedures. For example, during a cataract surgery a keratome (scalpel) having a blade ranging in thickness from about 0.1 mm to about 0.5 mm is used to form an elongated cut on the order of two (2) to six (6) mm in length at the base of the cornea. For a vitrectomy or retinal surgery the sclera of the eye is punctured with a trocar having an outside diameter in the range from about 0.64 to about 0.90 mm.

Such wounds are typically sealed using sutures. However, the use of sutures has some drawbacks. The placement of sutures inflicts trauma to the site, may serve as a locus for infection, and may lead to inflammation and vascularization, thereby increasing the chances of scarring. The use of sutures may also lead to uneven healing, resulting in astigmatism.

Accordingly, for some procedures such as sealing corneal cataract incisions, some surgeons prefer sutureless, self-sealing incisions because of the drawbacks of using sutures. However, sutureless incisions may leak and are points of potential ingress into the interior chamber by foreign bodies or contaminating fluids, which may cause complications such as endophthalmitis.

A potential alternative to either sutured or sutureless closure of ophthalmic wounds is the use of ophthalmic sealants. Various types of sealants have been proposed for sealing ophthalmic wounds. For example, the use of cyanoacrylates and fibrin sealants to seal ophthalmic wounds has been proposed.

Yet another class of ophthalmic sealants is a two-part hydrogel that is generally formed by reacting a component having nucleophilic groups with a component having electrophilic groups. The electrophilic groups of one component are capable of reacting with the nucleophilic groups of the other component to form a crosslinked network via covalent bonding. Kodokian et al. (copending and commonly owned U.S. Patent Application Publication No. 2006/0078536) describes hydrogel tissue adhesives formed by reacting an oxidized polysaccharide with a water-dispersible, multi-arm polyether amine. These adhesives provide improved adhesion and cohesion properties, crosslink readily at body temperature, maintain dimensional stability initially, do not degrade rapidly, and are nontoxic to cells and non-inflammatory to tissue.

Regardless of the type of sealant utilized there still remains a need for a dispensing device able to place microliter quantities of the selected sealant at the site of the wound.

In view of the foregoing it is believed advantageous to provide a dispensing device that is able to be easily handled by an operator and able to dispense microliter quantities of a material, such as a sealant, at any selected site of a wound. Thus, for use at the site of a keratome incision, it is believed advantageous that the dispensing device should be able to dispense a material either directly on top of the corneal slice or only to the cut surfaces inside the edges of the slice. For vitrectomy or retinal surgeries the dispensing device should be able advantageously to place material into the puncture site with a minimum of stretching of the tissue matter of the sclera.

### SUMMARY OF THE INVENTION

The present invention is directed toward a hand-held dispensing device for dispensing microliter quantities (on the order of about three (3) microliters) of one or two materials, such as the components of an adhesive sealant, into or onto the site of a wound, such as slice or a puncture wound.

The dispensing device includes a support platform having a pair of finger gripping surfaces thereon. The support platform carries a first and a second hollow (preferably tubular) member.

In one aspect of the invention each hollow member has a predetermined maximum outside dimension (diameter) that is not greater than about 0.4 mm, and preferably that lies in the range from about 0.3 mm to about 0.4 mm. When the pair of hollow members is positioned on the platform a circumscribing circle centered on a point between the hollow members at their discharge ends has a diameter that is not greater than about 0.8 mm, and preferably is in the range from about 0.6 mm to about 0.8 mm. So sized, both of the hollow members are insertable either into a space defined between the first and second edges of a site of a slice wound or into a site of a puncture wound. In either instance the insertion of the hollow members of the dispensing device into the wound occurs with minimal or little disruption (i.e., less than undue disruption) of any tissue matter surrounding the site.

Each hollow member has a predetermined largest inside dimension (diameter) lying in the range from about 0.2 mm to about 0.3 mm. A plunger is disposed in each hollow member with each plunger having a predetermined largest outside dimension (diameter) that is sized for compatible receipt within its associated hollow member. In accordance with another aspect of the present invention the largest outside dimension of each compatibly sized plunger is in a range from about eighty percent (80%) to about ninety-five percent (95%) of the largest inside dimension of a hollow member in which it is disposed. The relative sizing of each hollow member and its compatibly sized plunger permits the dispensing device to operate in a seal-less manner to efficiently deliver material to a wound site.

An actuator having a thumb actuating surface thereon is operatively engagable with each plunger and movable with respect to the support platform through a maximum actuating stroke defined between a fully extended position and a fully closed position. Movement of the actuator through an actuating stroke displaces each compatibly sized plunger with respect to the hollow member in which it is disposed from a first, loaded, position to a second, dispensed, position.

When in the fully extended position a predetermined maximum finger span is defined between the actuating surface of the actuator and the finger gripping surfaces. In accordance with another aspect of the present invention the maximum finger span for a dispensing device having first and second hollow members each with a compatibly sized plunger received therein is not greater than about 150 mm, and more preferably, lies in a range from about 49 mm to about 105 mm.

The various aspects of the present invention may be arranged in a single dispensing device in any combination. Thus, in accordance with the present invention, a dispensing device with a finger span in the recited range and with two hollow members each having a predetermined maximum outside dimension that lies in the range from about 0.3 mm to about 0.4 mm and each having a compatibly sized plunger the largest outside dimension of which is about eighty percent (80%) to about ninety-five percent (95%) of the inside dimension of its associated hollow member is operative to deliver a total volume (from both hollow members) of material in the range from about 0.5 to about 10 microliters with a delivery efficiency of at least about sixty (60%). Each hollow member would deliver material in the range from about 0.25 to about 5.0 microliters with the delivery efficiency of at least about sixty (60%).

### BRIEF DESCRIPTION OF THE DRAWINGS

The invention will be more fully understood from the following detailed description taken in connection with the accompanying drawings, which form a part of this application, and in which:

FIG. 1 is a diagrammatic exploded view of a seal-less, hand-held dispensing device for dispensing microliter quantities of a first and a second material at a predetermined site in accordance with the present invention;

FIG. 2 is a side elevation view of an assembled dispensing device shown in FIG. 1 with a portion of a finger cradle broken away;

FIG. 3A is a section view of the assembled dispensing device taken along section lines 3A-3A in FIG. 2, while FIG. 3B is an enlarged front elevation view of the assembled dispensing device taken along view lines 3B-3B in FIG. 2, with some structural details of the end of the device omitted for clarity; and

FIGS. 4A and 4B are stylized diagrammatic views illustrating the operation of a seal-less dispensing device of the present invention as a plunger is displaced from its loaded toward its dispensed position.

### DETAILED DESCRIPTION OF THE INVENTION

Throughout the following detailed description similar reference numerals refer to similar elements in all Figures of the drawings. It should be understood that various details of the structure and operation of the present invention as shown in various Figures have been stylized in form, with some portions enlarged or exaggerated, all for convenience of illustration and ease of understanding.

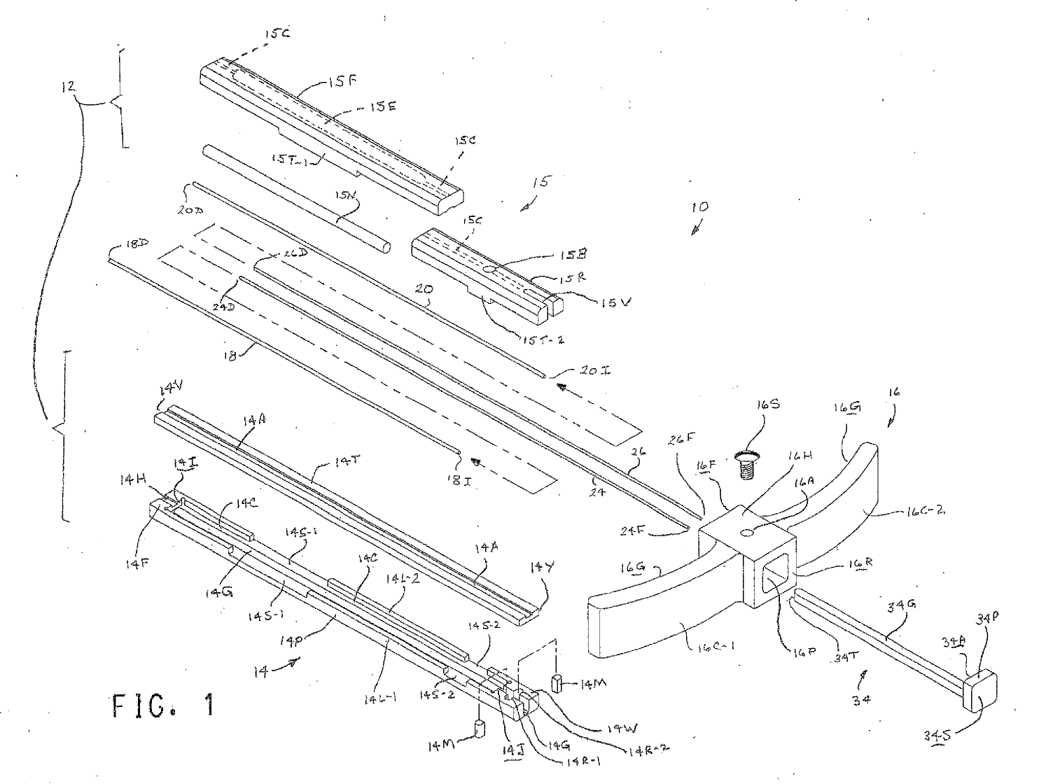

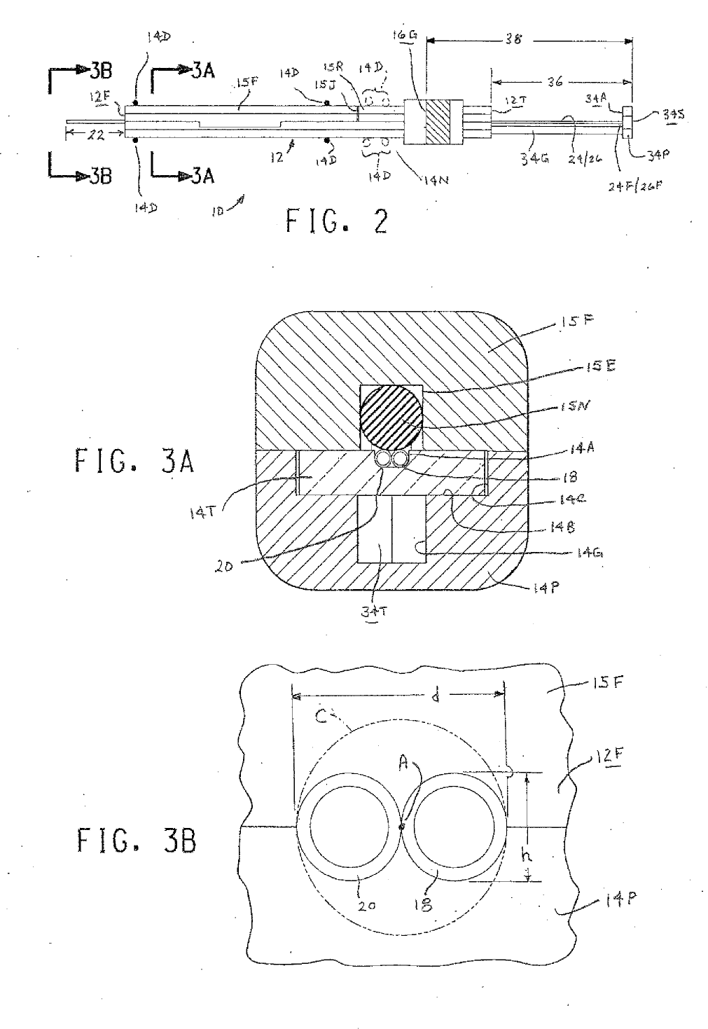

With reference to the drawings FIG. 1 shows an exploded view of a seal-less hand-held dispensing device generally indicated by the reference character 10 in accordance with the present invention. FIG. 2 is a side elevation view of the dispensing device 10 in its fully assembled configuration.

The dispensing device 10 is useful for dispensing microliter quantities of one or two material(s) into or onto a predetermined site. The dispensing device 10 is believed particularly useful in dispensing microliter quantities of a two-component medical adhesive sealant into the site of a wound, such as the type of wound produced during ophthalmic surgery. More specifically, the invention is adapted to introduce a two-component medical adhesive sealant, such as the hydrogel tissue adhesive disclosed in U.S. Patent Application Publication No. 2006/0078536 (Kodokian et al.), into an elongated slice wound such as produced by a keratome (scalpel) and/or a puncture wound such as produced by a trocar.

The dispensing device 10 includes a housing, or support platform, generally indicated by the reference character 12. The support platform 12 is shown in the exploded condition in FIG. 1 and in its assembled condition in FIG. 2. The platform 12 has a leading, or forward, end surface 12F (FIG. 2) and a trailing, or rear, end surface 12T. The platform 12 includes a base generally indicated by the reference character 14, a cover generally by the reference character 15, and a gripping handle generally by the reference character 16. The base 14 carries a first and a second hollow member 18, 20, respectively. Each hollow member 18, 20 has a discharge end 18D, 20D and an opposite, interior, end 181, 201, respectively. Each hollow member 18, 20 is preferably cylindrically tubular in form having a generally circular cross section in a plane perpendicular to it axis. Each hollow member 18, 20 is sized as will be described. As seen from FIG. 2 the discharge end 18D, 20D of each respective hollow member 18, 20 extends a predetermined overhang distance 22 beyond the forward surface 12F of the support platform 12.

Each hollow member 18, 20 is operative to carry and to dispense a predetermined desired volume of the same or a different selected material into or onto a desired site at which the respective discharge ends 18D, 20D of the members 18, 20 are placed. In one preferred instance each of the hollow members 18, 20 serves to dispense a different component of a two-component adhesive into or onto a slice wound or a puncture wound produced during surgery. However, it should be understood that the dispensing device 10 may advantageously be used to introduce a single material to a desired site through one or both of the tubular members 18, 20.

The gripping handle 16 preferably takes the form of a palmer pinch grip whereby an operator is able easily to grasp, to manipulate and to operate the dispensing device 10. The gripping handle 16 includes a central hub 16H having a forward surface 16F and a rear surface 16R. The hub 16H carries a pair of finger cradles 16C-1, 16C-2. Each cradle exhibits a finger-gripping surface 16G. Each finger-gripping surface 16G preferably has a generally rounded contour, although any alternative configuration of the gripping surfaces may be used. The hub 16H has a central passage 16P that extends completely therethrough. The gripping handle 16 is mounted on and secured to the cover 15 of the support platform 12 via a set screw 16S. The set screw 16S extends through an aperture 16A provided in the hub 16H and is threaded into a bore 15B formed in the cover 15, as will be discussed.

Each hollow member 18, 20 receives a respective plunger 24, 26. Each plunger 24, 26 has a predetermined largest outside dimension and a cross section configuration that makes each plunger compatible for receipt within the inside dimension and the cross section, respectively, of the complementarily sized hollow member 18, 20 in which it is disposed.

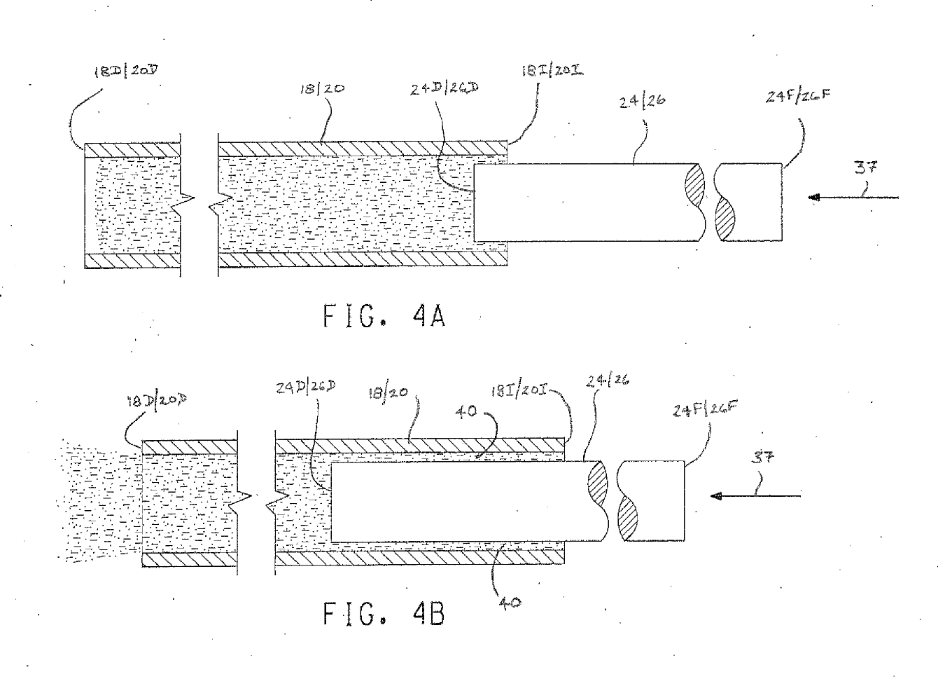

Accordingly, each plunger 24, 26 is preferably cylindrically tubular in form and is compatibly sized with respect to its associated hollow member, as will also be discussed. Each plunger 24, 26 has a preferably planar dispensing end surface 24D, 26D (best seen in FIGS. 4A, 4B) that is received within the compatible hollow member 18, 20. The opposite end of each plunger 24, 26 presents a preferably planar force-receiving surface 24F, 26F. Each plunger 24, 26 should preferably have a length dimension that is greater than the length of the member 18, 20 in which it is disposed, thereby to facilitate the charging of a material into and the dispensing of a material from the hollow member, as will be described. Each plunger 24, 26 is movable within its respective hollow member 18, 20 from a first, loaded, position to a second, dispensed, position.

A tubular member 18, 20 is individually charged with a material to be dispensed using its respective plunger 24, 26. Each plunger 24, 26 is completely inserted through its respective member 18, 20 such that its dispensing end 24D, 26D projects beyond the discharge end 18D, 20D of the member. Owing to the length of a plunger 24, 26 with respect to its respective associated member 18, 20, even when a plunger projects beyond the discharge end a portion adjacent to its force-receiving end 24F, 26F is still able to be manipulated at the interior end 18I, 20I of the hollow member 18, 20. The discharge end 18D, 20D of each hollow member 18, 20 is immersed into a pool of material. The plunger is then pulled into the member by grasping the plunger near its force-receiving end and drawing the dispensing end 18D, 20D of the plunger back into the hollow member 18, 20. During this movement capillary action combined with a siphoning action draw a volume of material into the hollow member. When each hollow member 18, 20 is charged with a desired initial volume of material the force-receiving end 24F, 26F of each plunger 24, 26 projects a predetermined distance rearwardly from the interior end 181, 201 of the respective hollow member 18, 20.

The volume of material that is charged is determined by the distance that each plunger 24, 26 is drawn back into its hollow member 18, 20. Of course, the volume of material initially loaded into a member should exceed the volume of material that is desired to be expelled into a wound site. The position of the dispensing end of a plunger with respect to the hollow member in which it is disposed when the member is charged to the desired initial volume defines the loaded position of the plunger.

The term “plunger length” is used herein to mean that distance that a given sized plunger must be displaced from its loaded position with respect to the compatibly sized hollow member in which it is disposed in order to expel a desired predetermined volume of material from the hollow member. The position of the dispensing end of a plunger with respect to its hollow member after the plunger is advanced the necessary plunger length defines the dispensed position of the plunger.

Motive force is imparted to the force-receiving ends 24F, 26F of the projecting lengths of the plungers 24, 26 to displace the plungers from the loaded to the dispensed positions using an actuator 34 (FIG. 1). The actuator 34 includes an actuating pad 34P having a thumb-actuating surface 34S and an opposed abutment surface 34A. A guide shaft 34G is secured to the abutment surface 34A. The guide shaft 34G has a tapered front end 34T. The guide shaft 34G is slidably received by the base 14 of the support platform 12 and guides the movement of the actuator 34 through its actuating stroke with respect to the platform 12.

The length 36 (FIG. 2) of the actuating stroke of the actuator 34 in any given operative instance is determined upon the plunger length, which in turn, is governed by the volume of the material desired to be dispensed from the hollow members 18, 20 by the advancement of the plungers 24, 26 therethrough.

As each plunger is moved from its loaded position toward its dispensed position the dispensing end of the plunger advances through its associated hollow member, thereby pushing the material present in the hollow member toward the discharge end of the hollow member. Owing to the relative dimensions of each plunger with respect to its associated hollow member advancement of the plunger is accomplished in a seal-less, self-sealing fashion (as discussed in connection with FIG. 4). By “seal-less” or “self-sealing” (or similar terms) it is meant the plunger does not carry a sealing member or gasket which is able to contact in wiping engagement against the interior surface of the hollow tubular member in which the plunger is disposed.

In general, the device 10 is operated by placing the finger pads of the index finger and middle finger of an operator on a respective gripping surface 16G and the thumb of the operator on the thumb-actuating surface 34S. As the thumb and fingers are brought together in a pinching movement the abutment surface 34A of the actuator pad 34P is brought to bear against the projecting force-receiving ends 24F, 26F of the plungers 24, 26. This action imparts motive force (diagrammatically indicated by the reference character 37, FIGS. 4A and 4B) to the plungers 24, 26, thereby advancing the plungers 24, 26 through the hollow members 18, 20 and moving the discharge ends of the plungers from their loaded toward their dispensed positions.

The disposition of these various operative parts of the dispensing device 10 at the beginning of an actuating stroke 36 of the actuator is shown in FIG. 2. The actuating stroke 36 terminates when the abutment surface 34A of the actuator contacts against the rearwardmost feature of the support platform 12, which, in the assembly illustrated in FIG. 2, is defined by the trailing surface 12T of the support platform 12.

The term “finger span” is used herein to denote the maximum distance 38 between the gripping surfaces 16G of the cradles 16C and the thumb-actuating surface 34S of the actuator 34 when the actuator 34 is received in the base 14. The finger span 38 of the dispensing device must be larger than the maximum actuating stroke of the actuator. As will be developed the dispensing device 10 in accordance with the present invention is configured such that the maximum extent of the finger span 38 falls within certain predetermined limits.

CONSTRUCTION DETAILS The construction of one implementation of the dispensing device 10 may be understood in more detail from the exploded view shown in FIG. 1. The base 14 of the support platform 12 includes a generally planar baseplate member 14P having a relatively thick front and back walls 14F, 14W respectively, and upstanding lateral sidewalls 14L-1, 14L-2. The sidewalls 14L-1, 14L-2 are interrupted by axially spaced front and rear pairs of slots 14S-1, 14S-2, respectively. The surfaces of the respective front and back walls 14F, 14W of the baseplate 14P form part of the respective forward and trailing surfaces 12F, 12T (FIG. 2) of the support platform 12.

A generally rectangular cavity 14C is formed in the baseplate 14P. The cavity 14C is bounded by the lateral sidewalls 14L-1, 14L-2, together with the inside surface 14I of the front wall 14F and the inside surface 14J of the back wall 14W. The corners of the cavity 14C are machined with rounded contours to prevent stress formation. The front wall 14F is interrupted by an axially extending planar shelf 14H that communicates with the cavity 14C.

The bottom surface 14B (FIG. 3A) of the cavity 14C has a guideway 14G formed therein. The guideway 14G extends axially from the inside surface 14J of the front wall 14F along the entire remaining length of the baseplate 14P. The guideway 14G extends completely through the back wall 14W. Recesses 14R-1, 14R-2 (FIG. 1) are formed in the back wall 14W and flank the guideway 14G. Each recess 14R-1, 14R-2 receives an elastomeric frictional spacer 14M.

A tube support tray 14T having a central, axially extending, open-ended channel 14A is supported on the bottom 14B of the cavity 14C. The tray 14T has a front end 14V and an interior end 14Y. When the device 10 is assembled the sides of the tray 14T are confined in the cavity 14C on the baseplate 14P by the upstanding sidewalls 14L-1, 14L-2. In addition, the front end 14V of the tray 14T abuts the inside surface 141 of the front wall 14F while the interior end 14Y abuts the inside surface 14J of the rear wall 14W.

The hollow members 18, 20 are held in abutting side-by-side relationship in the channel 14A on the tray 14T. The bottom corners of the channel 14A are slightly rounded to accommodate the contour of the members 18, 20. The forward portions of the hollow members 18, 20 are supported on the shelf 14H as they extend through and beyond the front wall 14F of the baseplate 14.

The baseplate 14P and tray 14T are overlaid by a front cover piece 15F and a rear cover piece 15R. The exterior surface of the front cover piece 15F and the front surface of the baseplate 14P cooperate to define the forward surface 12F of the platform 12. Similarly, the exterior surface of the rear cover piece 15R and the rear surface of the baseplate 14P cooperate to define the trailing surface 12T of the platform 12. The bore 15B that accepts the set screw 16S extends through the rear cover piece 15R. A viewing slot 15V is defined at the back end of the rear cover piece 15R.

Both the front and rear cover pieces 15F, 15R have a pair of depending tabs 15T-1, 15T-2, respectively. When assembled the tabs 15T-1, 15T-2 are received in the respective front and rear pairs of slots 14S-1, 14S-2 on the baseplate 14P.

A shallow channel 15C runs axially along the undersurface of each cover piece 15F, 15R. The channel 15C is positioned in the cover pieces so that it aligns with the channel 14A in the tray 14T when the covers 15F, 15R are placed over the base 14. A region of the channel 15C on the underside of the front cover piece 15F is enlarged, as at 15E (see also FIG. 3A), to accept an elastomeric cushion 15N (FIG. 3A).

ASSEMBLY Prior to the assembly of the device 10 the hollow members 18, 20 should be charged with material to be dispensed (as described above)

Once the elastomeric frictional spacers 14M have been inserted into the recesses 14R-1, 14R-2 flanking the guideway 14G the tray 14T is laid onto the bottom 14B of the cavity 14C. The rear cover piece 15R is then mounted onto the baseplate 14P by inserting the tabs 15T-2 on the sides of the rear cover piece 15R into the corresponding slots 14S-2 in the baseplate 14P. The channel 15C in the underside of the rear cover piece 15R aligns with the portion of the channel 14A in the baseplate 14P.

Next the handle 16 is put into place. The trailing end 12T of the baseplate subassembly (comprising the baseplate, tray and rear cover piece) is inserted (from the front surface 16F) into the central passage 16P of the hub 16H. The baseplate subassembly is closely sized to fit snugly within the passage 16P in the hub 16H. The baseplate subassembly is advanced into the passage 16P until the bore 15B in the rear cover piece 15R registers with the aperture 16A of the hub 16H. The parts are secured in their relative positions by the set screw 16S. At this point it is convenient to position the resilient bands 14D (FIG. 2) temporarily around the rear cover piece 15R and baseplate 14P. This temporary disposition of the bands 14D is suggested by the dashed lines at location 14N in FIG. 2.

Using a path substantially parallel with the surface of the tray 14T and leading with the force receiving ends 24F, 26F of the plungers 24, 26, the hollow members 18, are inserted through the aligned channels 14A, 15C in the conjoined tray 14T and cover piece 15R. Using the viewing slot 15V the hollow members 18, 20 are advanced until their interior ends 181, 201 are aligned with the interior end 14Y of the tray 14T. This aligns the interior ends 18I, 20I against the surface 14J of the back wall 14W. The hollow members 18, 20 are then fully seated in the channel 14A and the plungers 24, 26 project past the rear surface 16R of the hub 16. Since the shelf 14H is substantially coplanar with the channel 14A of the tray 14 the discharge ends 18D, 20D of the members 18, 20 extend the desired overhang distance 22 (FIG. 2) beyond the forward surface 12F of the support platform 12.

Next, with the cushion 15N in place in the enlarged region 15E, the front cover piece 15F is mounted onto the baseplate 14P by placing the tabs 15T-1 into the corresponding slots 14S-1 in the baseplate 14P. The cushion 15N bears against the hollow members 18, 20 (FIG. 3A) to bias them in position in the channel 14C of the tray 14Y. The cushion 15N also prevents the hollow members 18, 20 from being ejected from the device 10 as the actuator 34 displaces each plunger through its respective hollow member. The resilient bands 14D are then advanced from their temporary disposition to respective positions near the leading end surface 12F and near the joint 15J (FIG. 2) between the front and rear cover pieces 15F, 15R, respectively. Of course, any other suitable expedient may be used to attaché the cover piece 15F to the baseplate 14P.

Finally, the tapered end 34T of the actuator 34 is inserted into the opening of the guide channel 14G in the back wall W of the baseplate 14P. The actuator 34 is inserted into the guide channel until the abutment surface 34A thereof bears against the force-receiving surfaces 24F, 26F of the projecting plungers 24, 26. The sides of the actuator shaft 34G contact against the spacers 14M in the baseplate. In operation, the spacers 14M serve to frictionally oppose motion of the guide shaft 34G with respect to the baseplate 14, thus enhancing an operator's sense of tactile control of the device 10 through the actuating stroke.

CONSTRUCTION MATERIALS In the preferred implementation the baseplate 14P, the front cover 15F, and the rear cover 15R are machined from aluminum. The tray 14T is preferably made of glass owing to the ability to precisely machine the channel 14A therein, but could be made of any a hard, rigid, stable material. The spacers 14M and the cushion 14N are formed of any suitable resilient elastomeric material, preferably an inert, sterilizable material such as a fluoroelastomer material sold by E.I. du Pont de Nemours under the trademark VITON®. The hollow members 18, 20 are preferably glass capillaries, most preferably fused silica with a polyimide coating. Suitable capilliaries for use in the device are available from Polymicro Technologies, a subsidiary of Molex Corp., as TSP standard polyimide coated capillaries. The plungers 24, 26 are made of stainless steel wire. The set screw 16S is also stainless steel. The actuator 34 is preferably made of high strength carbon steel, such as stainless steel. The hub 16 is machined from aluminum, but could also be made by injection molding from a suitable plastic material polycarbonate, polystyrene or acrylic plastic, if desired. It should be noted that the device 10 may be made as a disposable item by fabricating it (other than the capillary tubes and the plungers) from an injection molded plastic material.

SIZING OF HOLLOW MEMBERS As mentioned earlier the dispensing device 10 in accordance with the present invention is sized and configured to enable an operator to introduce single-handedly one or two different material(s) (such as two adhesive components) into certain sized puncture and/or slice wounds. The dispensing device of the present invention is believed to find particular utility for use with slice and/or puncture wounds of the type produced by a keratome or a trocar during ophthalmic surgery. Since, as noted, each hollow member 18, 20 is preferably implemented in the form of hollow cylindrical tube the largest outside diameter dimension of the hollow members 18, 20 is a critical parameter in sizing the device for insertion into either a slice or a puncture wound.

As an example, a keratome having a blade width on the order of three (3) mm and thickness along the blade ranging from about 0.1 mm to about 0.5 mm would typically produce an elongated slice wound in the cornea of the eye of about two (2) to six (6) mm in length (“L”) with a predetermined distance (“H”) defined between the edges of the slice on the order not less than about 0.5 mm, typically in the range from about 0.3 to about 0.5 mm.

Accordingly, a dispensing device 10 amenable for use for dispensing a material into a slice wound of this size range requires hollow members 18, 20 with an outer diameter (“o.d.”) not greater than about 0.4 mm, preferably an outer diameter in the range from about 0.3 mm to about 0.4 mm, and more preferably, an outer diameter of about 0.3 mm. Hollow members 18, 20 with such outer diameter dimensions would present a height dimension “h” (FIG. 3B) that is substantially the same as the height dimension “H” of the wound being dressed. Thus, a device 10 wherein each tube 18, 20 has an outer diameter of about 0.3 mm is insertable into a space defined between the first and second edges of a slice wound with substantially minimal, if any, disruption to the tissue matter surrounding the site. If each tube 18, 20 has an outer diameter of about 0.4 mm, the device 10 is insertable into a wound with such edge spacing without undue disruption of any tissue matter surrounding the site. As used herein the term “undue disruption” means contact between the hollow members and the tissue in a way that does not injure, damage or tear the tissue.

As another example, a twenty-three (23) gauge trocar used for retinal surgery produces a puncture wound in the sclera of the eye having a maximum diameter (“D”) on the order of 0.64 mm, while the puncture wound produced by a twenty (20) gauge trocar would produce a puncture wound having a maximum diameter (“D”) almost fifty percent larger (i.e., on the order of 0.90 mm).

As seen in FIG. 3B, at their discharge ends the hollow member 18, 20 define a circumscribing circle C centered on a point A between the hollow members 18, 20 has a diameter “d”. Hollow members 18, 20 each with an outer diameter of about 0.3 mm cooperatively produce a circumscribing circle C with a diameter “d” that is on the order of about 0.6 mm. Members 18, 20 with such a size are thus amenable for use for dispensing a material into puncture wounds with diameters in the range from about 0.6 to about 0.9 mm (the wounds produced by a twenty gauge through a twenty-three gauge trocar) with substantially minimal disruption of any tissue matter surrounding the site.

A dispensing device 10 wherein each tube 18, 20 has an outer diameter not greater than about 0.4 mm, preferably each with an outer diameter in the range from about 0.3 mm to about 0.4 mm, and more preferably, each with an outer diameter of about 0.3 mm, is useful to treat a puncture wound with a diameter on the order of 0.9 mm (as produced by a twenty-three gauge trocar). Such a device would define a circumscribing circle C with a diameter “d” not greater than about 0.8 mm and in the range from about 0.6 mm to about 0.8 mm. This device would be amendable for use with substantially minimal disruption of any tissue matter surrounding the site.

COMPATIBLE PLUNGER SIZING From the immediately preceding discussion it should be appreciated that in order to be insertable into either a slice or a puncture wound of the type under discussion without unduly disrupting any tissue matter surrounding the site of the wound, each tube 18, 20 of the dispensing device 10 has an outer diameter not greater than about 0.4 mm, preferably each with an outer diameter in the range from about 0.3 mm to about 0.4 mm.

A glass capillary tube with an outer diameter of 0.3 mm has an inside diameter of about 0.2 mm. A glass capillary tube with an outer diameter of about 0.4 mm has an inside diameter of about 0.3 mm.

Each cylindrical plunger 24, 26 must have a predetermined largest outside dimension (i.e., diameter) that is sized for compatible receipt within an associated hollow capillary tube.

Tables I and II list, in the first columns of each, the outside diameter dimension of readily available wires that are sized compatibly for use as a plunger within glass capillary tubes having an inside diameter in the range from about 0.3 mm to about 0.4 mm. All wire sizes listed are available from Small Parts, Incorporated, c/o Amazon.com Incorporated, Seattle, Wash.

Each table also lists under a “Ratio” column the ratio of the outer diameter of each plunger wire with respect to the inside diameter of the glass capillary in which it is compatibly disposed.

The column headed “Plunger Length” in each Table sets forth, for each wire size, the distance with respect to a hollow member that a compatibly sized plunger must be displaced from its loaded to its dispensed position in order for a predetermined volume of material (in this case, about three microliters) to be expelled from the tube.

The column headed “Delivery Efficiency” measures the percentage of available material dispensed by a plunger when moved over its corresponding plunger length, relative to the volume of material within that length of tube. The “Delivery Efficiency” of a given tube/plunger is an important parameter to consider in preventing material waste. A “Delivery Efficiency” of about sixty percent (60%) is believed to be a reasonable standard.

TABLE I
Plunger compatible for glass capillary with 0.3 outside diameter
0.2 mm inside diameter

| Plunger | Plunger | Ratio (plunger o.d. | Delivery |
|---|---|---|---|
| o.d. (mm) | Length (mm) | to tube i.d.) (%) | Efficiency (%) |
| 0.1905 | 105 | 95.25 | 90.7 |
| 0.1778 | 121 | 88.50 | 79.0 |
| 0.1651 | 140 | 82.55 | 68.1 |
| 0.1524 | 164 | 76.20 | 58.1 |
| 0.1397 | 196 | 69.85 | 48.8 |
| 0.1270 | 237 | 63.50 | 40.3 |

TABLE II
Plunger compatible for glass capillary with 0.4 outside diameter
0.3 mm inside diameter

| Plunger | Plunger | Ratio (plunger o.d. | Delivery |
|---|---|---|---|
| o.d. (mm) | Length (mm) | to tube i.d.) (%) | Efficiency (%) |
| 0.2794 | 49 | 93.1 | 86.7 |
| 0.2540 | 59 | 84.7 | 71.7 |
| 0.2413 | 66 | 80.4 | 64.7 |
| 0.2286 | 73 | 76.2 | 58.1 |
| 0.2159 | 82 | 72.1 | 51.8 |
| 0.2032 | 94 | 67.7 | 45.8 |
| 0.1905 | 105 | 63.5 | 40.3 |

It is clear from an analysis of Tables 1 and 2 that for a dispensing device having hollow members with an inside diameter lying in the range from about 0.2 mm to about 0.3 mm to achieve a reasonable delivery efficiency of about sixty percent (60%) the outside diameter of a compatibly sized plunger should lie within a range from about 80% to about 95% of the largest inside dimension of a hollow member in which it is disposed. In addition to providing reasonable delivery efficiency a plunger within this recited range of the inside diameter of the hollow member enables the dispensing device to operate in a seal-less manner.

Seal-less operation of the dispensing device is believed clearly depicted in FIGS. 4A and 4B. As an actuating force 37 imposed on the force-receiving surface 24F/26F of the plunger 24/26 by the abutment surface of the actuator, the dispensing end 24D/26D of the plunger displaced with respect to the hollow member 18/20 from its loaded toward its dispensed position. Owing to the close sizing between the outside dimension of the plunger 24/26 and the inside dimension of the plunger respective compatibly sized plunger as the plunger is advanced through its respective member 18/20 an annulus 40 of material is defined between the confronting outer and inside surface. This annulus of material serves as a seal between these members.

Thus, the dispensing device in accordance with the invention is able to operate in a seal-less, self-sealing fashion, without the need for a sealing member or gasket contacting in wiping engagement against the interior surface of the member. This mode of operation minimizes the force required to advance the actuator through its actuating stroke.

FINGER SPAN In accordance with another aspect of the present invention the maximum finger span is not greater than about 150 mm, and more preferably, is in the range from about 50 to about 105 mm. The preferred range (about 50 to about 105 mm) would permit about 92% of the adult population to operate the dispensing device of the present invention in an ergonomically effective manner.

These percentages are based upon a study of actual hand dimensions by A. K. Agnihotri, et al, in the article titled “Determination of Sex by Hand Dimensions” in the Internet Journal of Forsenic Science 2006: Volume 1 Number 2. In this study the size range for hand length is 14.8 to 21.0 cm, where hand length is measured from the distal crease of the wrist joint to the tip of the middle finger. The defined ranges for finger span are determined by adjusting these hand lengths in accordance with a predetermined proportionality constant (approximately 0.65) relating finger span to hand length.

MAXIMUM DISPENSING CAPABILITY A dispensing device 10 with a finger span in the recited ranges and with two hollow members each having a predetermined maximum outside dimension that lies in the range from about 0.3 mm to about 0.4 mm and each having a compatibly sized plunger the largest outside dimension of which is about eighty percent (80%) to about ninety-five percent (95%) of the inside dimension of its associated hollow member is operative to deliver a total volume of material in the range from about 0.5 to about 10 microliters with a delivery efficiency of at least about sixty percent (60%). Thus, each hollow member would deliver material in the range from about 0.25 to about 5.0 microliters with a delivery efficiency of at least about sixty (60%) percent.

From the foregoing discussion it should be appreciated that the present invention provides a dispensing device 10 that may be easily grasped, manipulated and handled to permit single handed dispensing of a material at a predetermined site. The device 10 achieves these ends by optimizing a plurality of important structural parameters. The hollow members are dimensioned so as to be insertable into the site of a slice or a puncture wound with minimal or not more than undue disruption of the tissue surrounding the site. Each hollow member and its plunger are compatibly sized to insure a predetermine aliquot of materials is efficiently delivered, and which operates in a seal-less manner. Moreover, the device is sized to exhibit a maximum finger span that renders it operable by a majority of operators.

Those skilled in the art, having the benefits of the present invention as herinabove set forth may impart modifications thereto. Such modifications are to be construed as lying within the contemplation of the present invention, as defined by the appended claims.

What is claimed is:

1. A seal-less, hand-held dispensing device for dispensing microliter quantities of a material at a predetermined site, the dispensing device comprising:
a support platform, the support platform having a pair of finger gripping surfaces thereon;
a first and a second hollow member disposed on the support platform, each hollow member having a predetermined largest inside dimension lying in the range from about 0.2 mm to about 0.3 mm,
a plunger disposed in each hollow member, each plunger having a predetermined largest outside dimension that is sized for compatible receipt within its associated hollow member,
the largest outside dimension of each compatibly sized plunger being in a range from about eighty percent (80%) to about ninety-five percent (95%) of the largest inside dimension of a hollow member in which it is disposed; and
an actuator having a thumb actuating surface thereon, the actuator being operatively engagable with each plunger and movable with respect to the support platform through a maximum actuating stroke defined between a fully extended position and a fully closed position,
movement of the actuator through its actuating stroke displacing each compatibly sized plunger with respect to the hollow member in which it is disposed from a first, loaded, position to a second, dispensed, position,
when in the fully extended position a predetermined maximum finger span is defined between the actuating surface of the actuator and the finger gripping surfaces,
the maximum finger span for a dispensing device having first and second hollow members each with a compatibly sized plunger received therein being not greater than about 150 mm.

2. The dispensing device of claim 1 wherein the maximum finger span is in a range from about 50 mm to about 105 mm.

3. The dispensing device of claim 1 wherein both the hollow members and the plungers received therein are circular in cross section.

4. The dispensing device of claim 1 wherein hollow members are disposed in side-by-side relationship on the support platform.

5. The dispensing device of claim 4 wherein each hollow member has an axis therethrough, and wherein the axes of the hollow members are substantially parallel to each other.

6. The dispensing device of claim 1 wherein the hollow members overhang a predetermined distance beyond the support platform.

7. The dispensing device of claim 1 wherein
the inside dimension of both hollow members are equal to each other, and
the outside dimension of both plungers are equal to each other.

8. The dispensing device of claim 1 wherein the inside dimensions of the hollow members are different.

9. The dispensing device of claim 1 wherein each plunger has a length greater than the length of the hollow member in which it is received.

10. The dispensing device of claim 1 wherein the support platform further comprises a cover having a cushion disposed therein, the cushion contacted against the hollow members.

11. The dispensing device of claim 1 wherein the support platform includes a baseplate having a pair of resilient spacers, each spacer is disposed in frictional producing engagement with the actuator.
